(19) United States
(12) Patent Application Publication
(10) Pub. No.: US 2008/0206273 A1
(43) Pub. Date: Aug. 28, 2008

(54) Cosmetic Product Comprising a Seaweed Extract, in Particular Carrageen

(75) Inventors: Helen Ambrosen, Dorset (GB); Mark Constantine, Dorset (GB); Margaret Constantine, Dorset (GB)

Correspondence Address: WESTMAN CHAMPLIN & KELLY, P.A., SUITE 1400, 900 SECOND AVENUE SOUTH, MINNEAPOLIS, MN, 55402-3244, US

(73) Assignee: Cosmetic Warriors Ltd., Poole, Dorset (GB)

(21) Appl. No.: 11/794,077

(22) PCT Filed: Dec. 16, 2005
(86) PCT No.: PCT/GB05/04893

§ 371 Date: Dec. 7, 2007

(30) Foreign Application Priority Data
Dec. 23, 2004 (GB) 0428247.1

(51) Int. Cl. A61K 8/97; A61K 8/34

(52) U.S. Cl. 424/195.17; 514/772; 424/777; 424/725

(57) ABSTRACT

The present invention resides in a cosmetic product comprising a surfactant, glycerine monopropylene glycol, and a seaweed extract.

The present invention relates to a cosmetic product particularly a cosmetic product having the form of a jelly.

It is known to produce cosmetic jellies by mixing a surfactant with an extract of seaweed. Such products have been sold commercially but have not been widely adopted, at least in part due to the properties of the jelly. That is, the known cosmetic jellies have a semi-liquid form and when, for example, applied to the human body with water they break down into the consistency of a lumpy paste.

The present invention seeks to improve upon the previously known cosmetic jellies.

According to a first aspect of the present invention there is provided a cosmetic product comprising a surfactant, glycerine and monopropylene glycol. Preferably the product also contains a seaweed extract such as carageen.

According to a second aspect of the present invention there is provided a method of forming a cosmetic product comprising the steps of selecting the components of the product to include a surfactant, glycerine and monopropylene glycol. Preferably the method also contains the step of selecting the components of the product to include a seaweed extract such as carageen.

The inventors hereof have discovered that the inclusion of monopropylene glycol, mixed with the glycerine, has a remarkable effect upon the consistency of the jelly, resulting in the jelly having a durable and almost rubber-like texture. The jelly is much firmer than a conventional jelly and readily holds a shape imparted to it, for example by moulding. These effects are all the more surprising when the jelly contains a seaweed extract, since it was thought that such an extract would be destabilised by the presence of glycerine and monopropylene glycol.

The jelly of the present invention is particularly useful as a cosmetic product because it does not readily break-down in the way that a conventional cosmetic jelly does. It is therefore particularly suitable for use as a shower gel or body wash, where it can be applied in a somewhat similar fashion to a conventional bar of soap. The product is also suitable for use as, for example, a hair wash. Unlike the conventional cosmetic jellies, and conventional soap bars, the texture of the jelly of the present invention enables it to be torn; so that a small piece suitable for a single application is readily obtained. Indeed the jelly can be moulded in a shape which assists such tearing into individual applications.

The jelly of the present invention has the described semi-solid form at room temperature. It also has the advantage that it retains that form at reduced temperatures. That is, whereas a conventional cosmetic jelly placed in a domestic freezer and reduced to a temperature of say −18° C. to −20° C. becomes a frozen solid, the jelly of the present invention does not. Of course, glycol is known as an anti-freeze but it was none-the-less surprising that it resulted in the jelly of the present invention maintaining it's durable rubber-like texture at temperatures of around −20° C. As an attribute of a cosmetic product such as a shower gel this feature is of considerable interest since it enables a “frozen” shower gel to be provided. The effect of applying the cold jelly to the human body in the presence of warm water, as in a shower, is very stimulating and invigorating. A conventional jelly when reduced to −20° C. becomes frozen solid and is thus somewhat abrasive and harsh when applied to the human body. In contrast the jelly of the present invention, retaining it's rubber-like texture at that temperature, does not suffer that disadvantage.

Embodiments of the present invention will now be described by way of further example only.

A cosmetic jelly according to an embodiment of the present invention is formed from the following components, percentages given by weight:—

| | |
|---|---|
| Water | 35% |
| Surfactant | 30% |
| Glycerine | 25% |
| Monopropylene Glycol | 7% |
| Seaweed Extract | 1.5% |
| Other (eg fragrance, preservative) | 1.5% |

The preferred range, by weight, of water among the components used to form a jelly according to the present invention is 15% to 45%.

The preferred range, by weight, of surfactant among the components used to form a jelly according to the present invention is 20% to 40%.

The preferred range, by weight, of glycerine among the components used to form a jelly according to the present invention is 10% to 35%.

The preferred range, by weight, of monopropylene glycol among the components used to form a jelly according to the present invention is 3% to 35%.

The preferred range, by weight, of seaweed extract among the components used to form a jelly according to the present invention is 1% to 3%.

A cosmetic jelly according to another embodiment of the present invention is formed from the following components, percentages given by weight:—

| | |
|---|---|
| Water | 35.3% |
| Surfactant | 30.0% |
| Glycerine | 25.0% |
| Monopropylene Glycol | 7.0% |
| Seaweed Extract | 1.5% |
| Fragrance | 1.0% |
| Preservative | 0.2% |

In the embodiments given above the water can be replaced by any desirable infusion, for example an infusion of herbs and fruit juices.

As specific examples, the seaweed extract referred to in the embodiments may be carageen and the surfactant may be sodium laurate sulphide, sold under the trade name mannec. Methylparaben, sold under the trade name nipagin, may be used as a preservative. Thus, a specific embodiment of the present invention is formed from the following groups of components, percentages given by weight:—

Group A

| | |
|---|---|
| Water based infusion | 37.5% |

Group B

| | |
|---|---|
| Monopropylene Glycol | 10.0% |
| Nipagin | 0.2% |

Group C

| | |
|---|---|
| Mannec | 31.2% |

Group D

| | |
|---|---|
| Glycerine | 17.4% |
| Carageen | 1.7% |

Group E

| | |
|---|---|
| Fragrance | 2.0% |

The infusion of Group A may, for example, consist of (by weight) 31.5% water, 1% herb and 5% juice.

A method of forming a jelly according to the present invention and based upon the above stated groups of components is a s follows: —

(1) form the group A infusion

(2) warm the group B components together so as to dissolve the nipagin

(3) subsequently add together the group A, B and C components

(4) heat to 80° C.

(5) add the group D components, which have been pre-mixed to make a paste

(6) remove from heat and add the group E component

The above stated method is an example only. The method may be varied, especially dependent upon the specific components used. For example the step of heating to 80° C. is considered to be required due to the inclusion of carageen. With the use of other forms of seaweed extract heating above room temperature may not be required at all.

In terms of the final product a particularly advantageous cosmetic jelly according to the present invention includes approximately 17% glycerine by weight of the final product and 10% monopropylene glycol by weight of the final product.

In terms of the final product, desirably a cosmetic jelly according to the present invention includes 10% to 35% glycerine by weight of the final product and 3% to 35% monopropylene glycol by weight of the final product.

Further desirably, a cosmetic jelly according to the present invention in terms of the final product includes 20% to 40% surfactant by weight of the final product.

Beneficially, a cosmetic jelly according to the present invention in terms of the final product includes 1% to 3% seaweed extract by weight of the final product.

It is to be noted that whereas the use of a seaweed extract such as carageen is recited above, it is considered that other natural gums, not necessarily seaweed extracts may achieve the same result.

1. A cosmetic product comprising a surfactant, glycerine and monopropylene glycol.

2. A cosmetic product as claimed in claim 1, further comprising a seaweed extract.

3. A cosmetic product as claimed in claim 1, wherein the percentage by weight of surfactant is 20% to 40%.

4. A cosmetic product as claimed in claim 1, wherein the percentage by weight of glycerine is 10% to 35%.

5. A cosmetic product as claimed in claim 1, wherein the percentage by weight of monopropylene glycol is 3% to 35%.

6. A cosmetic product as claimed in claim 2, wherein the percentage by weight of seaweed extract is 1% to 3%.

7. A cosmetic product as claimed in claim 1, comprising approximately 17% glycerine by weight of the final product and 10% monopropylene glycol by weight of the final product.

8. A cosmetic product as claimed in claim 1, further comprising a fragrance.

9. A cosmetic product as claimed in claim 1, further comprising a preservative.

10. A cosmetic product as claimed in claim 1, wherein the surfactant is sodium laurate sulphide.

11. A cosmetic product as claimed in claim 2, wherein the seaweed extract is carageen.

12. A cosmetic product as claimed in claim 9, wherein the preservative is methylparaben.

13. A cosmetic product as claimed in claim 1, further comprising one or more herbs and a fruit juice.

14. A method of forming a cosmetic product comprising the steps of selecting the components of the product to include a surfactant, glycerine and monopropylene glycol.

15. A method as claimed in claim 14, further comprising the step of selecting a seaweed extract as a component of the product.

16. A method as claimed in claim 14, further comprising the step of including water in the amount 15% to 45% by weight among the components selected to form the product.

17. A method as claimed in claim 14, further comprising the step of including the surfactant in the amount 20% to 40% by weight among the components selected to form the product.

18. A method as claimed in claim 14, further comprising the step of including the glycerine in the amount 10% to 35% by weight among the components selected to form the product.

19. A method as claimed in claim 14, further comprising the step of including the monopropylene glycol in the amount 3% to 35% by weight among the components selected to form the product.

20. A method as claimed in claim 15, further comprising the step of including the seaweed extract in the amount 1% to 3% by weight among the components selected to form the product.

21. A method as claimed in claim 14, further comprising the step of selecting the surfactant to be sodium laurate sulphide.

22. A method as claimed in claim 15, further comprising the step of selecting the seaweed extract to be carageen.

23. A method as claimed in claim 14, comprising the steps of selecting the components of the product to be, percentages given by weight:—

| | |
|---|---|
| Water | 35% |
| Surfactant | 30% |
| Glycerine | 25% |
| Monopropylene Glycol | 7% |
| Seaweed Extract | 1.5% |
| Other components | 1.5%. |

24. A method as claimed in claim 23, comprising the step of selecting the said other components to include at least one of a fragrance and a preservative.

25. A method as claimed in claim 14, comprising the steps of selecting the components of the product to be, percentages given by weight:—

| | |
|---|---|
| Water | 35.3% |
| Surfactant | 30.0% |
| Glycerine | 25.0% |
| Monopropylene Glycol | 7.0% |
| Seaweed Extract | 1.5% |
| Fragrance | 1.0% |
| Preservative | 0.2%. |

26. A method as claimed in claim 23, further comprising substituting a water based infusion for the said water.

27. A method as claimed in claim 14, comprising the steps of selecting the components of the product to be the following groups of components, percentages given by weight:—
Group A

| | |
|---|---|
| Water based infusion | 37.5% |

Group B

| | |
|---|---|
| Monopropylene Glycol | 10.0% |
| Methylparaben | 0.2% |

Group C

| | |
|---|---|
| Sodium laurate sulphide | 31.2% |

Group D

| | |
|---|---|
| Glycerine | 17.4% |
| Carageen | 1.7% |

Group E

| | |
|---|---|
| Fragrance | 2.0%. |

28. A method as claimed in claim 26, further comprising the step of selecting the infusion to contain a herb, a juice and water.

29. A method as claimed in claim 27, further comprising the following steps:—
(1) forming the group A infusion
(2) warming the group B components together so as to dissolve the methylparaben
(3) subsequently adding together the group A, B and C components
(4) heating the added together group A, B and C components
(5) adding the group D components, which have been pre-mixed to make a paste
(6) removing from heat and add the group E component.
